(19) United States
(12) Patent Application Publication
(10) Pub. No.: US 2005/0203495 A1
(43) Pub. Date: Sep. 15, 2005

(54) Methods and devices for plasmon enhanced medical and cosmetic procedures

(75) Inventor: Henryk Malak, Ellicott City, MD (US)

Correspondence Address: Henryk Malak, 8444 High Ridge Road, Ellicott City, MD, 21043, US

(73) Assignee: American Environmental Systems, Inc.

(21) Appl. No.: 10/916,560

(22) Filed: Aug. 12, 2004

Related U.S. Application Data
(60) Provisional application No. 60/552,854, filed on Mar. 15, 2004.
(60) Provisional application No. 60/551,382, filed on Mar. 10, 2004.

(51) Int. Cl. A61B 18/20

(52) U.S. Cl. 606/9; 606/15

(57) ABSTRACT

Composition of methods and devices for surface plasmon resonance-enhanced medical and cosmetic procedures are disclosed. The invention relates to the use of a nonlinear surface plasmon resonance generation source and metal nanoparticles embedded to a body to enhance medical and cosmetic procedures in the body. The methods and devices in this invention can be applied for very effective three-dimensionally localized body surgery, tattoo removal, skin pigmentation removal, hair removal, drug delivery, photodynamic therapy, thrombosis, lithotripsy and cosmetic body treatment. The present invention relates also to a method of making temporary, semi-permanent and permanent tattoos with surface plasmon resonance technique.

### CROSS REFERENCE TO RELATED APPLICATIONS

This application is related to U.S. Provisional Patent Application No. 60/552,854 entitled “Plasmon Enhanced Laser Surgery and Methods Therefore” filed Mar. 15, 2004 and to U.S. Provisional Patent Application No. 60/551,382 entitled “Tattoo and Skin Pigmentation Removal with Plasmon Enhanced Nonlinear Optical Methods” filed Mar. 10, 2004, which are herein incorporated by reference.

### STATEMENT REGARDING FEDERALLY SPONSORED RESEARCH

There is NO claim for federal support in research or development of this product.

### REFERENCES CITED

The following are patents found that may be associated with this information.

### U.S. PATENT DOCUMENTS

U.S. Pat. No. 6,428,811 Aug. 6, 2002 West et al.
U.S. Pat. No. 6,530,944 Mar. 11, 2003 West et al.

### OTHER REFERENCES

Kochevar, I. E. “Biological Effects of Excimer Laser-Radiation” Proceedings of the IEEE, 80(6), pp. 833-837, (1992).
R. R. Anderson, J. A. Parrish, “Selective photothermolysis: precise microsurgery by selective absorption of pulsed radiation”, Science, 220, pp. 524-527 (1983).
McKenzie, G. P., Beck, C. M., Mitchell, J., et al. in Proceedings of SPIE, Thermal Treatment of Tissue: Energy Delivery and Assessment. “Confined Tissue Ablation for Vitrectomy: a study at FELIX” Ed. Ryan, T. P. Vol. 4247, pp. 229-237. Presented at Photonics West, SPIE, San Jose, Calif., (2001).
Navarro, L., Min, R. J., & Bone, C. (2001); Alster T, Apfelberg D B (eds). Cosmetic laser surgery. New York: John Wiley & Sons, Inc., Endovenous laser: A new minimally invasive method of treatment for varicose veins—preliminary observations using an 810 nm diode laser. Dermatologic Surgery, 27, 117-122, (1995).
Edwards, G., Logan, R., Copeland, M., et al. “Tissue ablation by a free-electron laser tuned to the amide II band.” Nature, 371(6496), pp. 416-9, (1994).
Uhlhorn, S. R., Mongin, D., Mackanos, M. A. & Jansen, E. D. in Tissue Interaction XII: Photochemical, Photothermal, and Photomechanical, Effects of IR wavelength on ablation mechanics: A study of acoustic signals. “Effects of IR wavelength on ablation mechanics: A study of acoustic signals” Ed. Jacques, S. L. Vol. 4257. Presented at Photonics West, SPIE, San Jose, Calif., (2001).
Auerhammer, J. M., Walker, R., van_der_Meer, L. & Jean, B. “Dynamic Behaviour of photoablation products of corneal tissue in the mid-IR: a study with FELIX” Applied Physics B, 68, pp. 111-119,(1999).
Schwettman, H. & Crosson, E. R. in Proceedings of SPIE, Biomedical Applications of Free-Electron Lasers. “Mid-infrared ablation with single high-intensity picosecond pulses (oral presentation only)” Ed. Edwards, G. S. & Sutherland, J. C. Vol. 3925. Presented at Photonics West, SPIE, San Jose, Calif., (2000).
Uhlhorn, S. R., London, R. A., Makarewicz, A. J. & Jansen, E. D. in Proceedings of SPIE, Laser Tissue Interaction XI: Photochemical, Photothermal, Photomechanical. “Hydrodynamic modeling of tissue ablation with a free-electron laser” Ed. Jacques, S. L. Vol. 3914A. Presented at Photonics West, SPIE, San Jose, Calif., (2000).
X. H. Hu, W. A. Woodenb, M. J. Cariveau, Q. Fan, J. F. Bradfield, G. W. Kalmusd, S. J. Vole, Y. Sun, Tattoo Removal in Micropigs with Low-energy Pulses from a Q-switched Nd:YAG Laser at 1064 nm. SPIE Proceedings, Vol. 4244 (2001)).
R. E. Fitzpatrick, M. P. Goldman, J. Ruiz-Esparza, “Use of the alexandrite laser (755 nm, 100 ns) for tattoo pigment removal in an animal model”, J. Am. Acad. Dermatol., 28,745-750 (1993).
T. J. Stafford, R. Lizek, J. Boll, O. T. Tan, “Removal of colored tattoos with the Q-switched Alexandrite laser”, Plus. Reconstroc. Surg., 95, pp. 313-320 (1995).
R. G. Wheeland, “Clinical uses of lasers in dermatology”, Lasers Surg. Med. 16, pp. 2-23 (1995).
N. Bloembergen, “Laser-induced electric breakdown in solid”, IEEE. J. Quan. Electron., 10, pp. 375-386 (1974).
D. Stern, R. W. Schoenlein, C. A. Puliafito, E. T. Dobi, R. Biringruber, J. G. Fujimoto, “Corneal ablation by nanosecond, picosecond, and femtosecond lasers at 532 and 625 nm”, Arch. Ophthalrnol., 107,587-592 (1989).
F. Johnson and M. Dovale, Intense pulsed light treatment of hirsutism: case reports of skin phototypes V and VI J. Cutan Laser Therapy 1: 233-237, (1999).
Tse Y. Hair removal using a pulsed-intense light source. Dermatol Clin, 17: 373-85, 1999.
M. Kerker, “Optics of colloid silver”, J. Colloid Interface Sci. 105, 298 (1985).
Lakowicz et al, “Intrinsic fluorescence from DNA can be enhanced by metallic particles”, Biochem. Biophys. Res. Comm. 286, 875 (2001).
Gryczynski et al., “Multiphoton excitation of fluorescence near metallic particles: enhanced and localized excitation”, J. Phys. Chem. B, 106, 2191 (2002).
M. Moskovits: Rev. Mod. Phys. 57, 783 (1985); T. L. Haslett, L. Tay, M. Moskovits: J. Chem. Phys. 113, 1641 (2000).
K. Kneipp, Y. Wang, H. Kneipp, L. T. Perelman, I. Itzkan, R. R. Dasari, M. S. Feld: Phys. Rev. Lett. 78, 1667 (1997).
Ditlbacher H. et al., Appl. Phys. B 73, 373-377 (2001).
Hirsch et al., PNAS, 100, 13549-13554 (2003), ultrasound (S. Coyle, et al., Phys. Rev. Let. 87(17), 176801, (2001).
George H. Patterson and David W. Piston, Photobleaching in Two-Photon Excitation Microscopy, Biophys J, Vol. 78, No. 4, p. 2159-2162 (2000).
S. Coyle, et al., Phys. Rev. Let. 87(17), 176801, (2001).

### FIELD OF THE INVENTION

This invention relates to nanotechnological and medicinal methods and devices used for enhanced body surgery, tattoo and skin pigmentation removal, and other body treatments.

### BACKGROUND OF THE INVENTION

In recent years there has been great interest in the use of laser cutting and ablation during surgery. Since the first laser was used in surgery more than 30 years ago, lasers have been employed extensively in the clinical environment. Although studies have been made into the relative efficiencies of various laser sources when applied to surgery, the ablation mechanisms are not fully understood. Pulsed and CW laser sources are used in a variety of clinical situations, although their side effects can severely limit their application to some areas.

Lasers are used in surgery as they allow for precise, clean cutting of tissues, with minimal collateral damage and with reduced bleeding. This allows for far more precise, accurate surgery to be undertaken by the surgeon, and in some cases surgery that would otherwise be impossible can be performed. As well as the very well documented ophthalmic and cosmetic applications of lasers, they are also finding uses in orthopedics and dentistry. There are problems in using lasers in medicine. Ultraviolet lasers, which cut very cleanly with little thermal or mechanical collateral damage, can cause DNA damage to tissue (Kochevar, I. E. (1992) “Biological Effects of Excimer Laser-Radiation” Proceedings of the Ieee, 80(6), pp. 833-837.), with obvious carcinogenic effects resultant from this. In addition, they are often bulky, expensive laser systems, which require a high level of maintenance.

In contrast, infrared lasers are usually solid state, relatively low cost systems that require far less maintenance than a typical UV system. In addition, IR systems lack the carcinogenic potential of the IR systems, meaning that they are safer for the surgeon and patient. However in fact, they do not, at present, cut as cleanly as a UV system. Because, their cutting action is purely photothermal and photomechanical, rather than the photochemical action partially employed by the UV systems.

Thermal denaturation weakens the structural matrix of the tissue. Explosive transition of tissue water to high-pressure vapor then ruptures the structural matrix, propelling the ablated material from the site of irradiation. In situations where there is a tissue-air boundary, this second mechanism is clearly seen as an ablation plume; this has been documented in various studies (R. R. Anderson, J. A. Parrish, “Selective photothermolysis: precise microsurgery by selective absorption of pulsed radiation”, Science, 220, pp. 524-527 (1983); McKenzie, G. P., Beck, C. M., Mitchell, J., et al. (2001a) in Proceedings of SPIE, Thermal Treatment of Tissue: Energy Delivery and Assessment. “Confined Tissue Ablation for Vitrectomy: a study at FELIX” Ed. Ryan, T. P. Vol. 4247, pp. 229-237. Presented at Photonics West, SPIE, San Jose, Calif.; Navarro, L., Min, R. J., & Bone, C. (2001); Alster T, Apfelberg D B (eds). Cosmetic laser surgery. New York: John Wiley & Sons, Inc., (1995). Endovenous laser: A new minimally invasive method of treatment for varicose veins—preliminary observations using an 810 nm diode laser. Dermatologic Surgery, 27, 117-122). It is generally accepted that there is a trade-off between thermal damage to surrounding tissue caused by longer pulses (generally >100 ms), and mechanical damage caused by short pulses (<1 ms). Explosive transition ablation produces a region of heat-affected tissue, as its mechanism is entirely thermal. A significant area surrounds the site of ablation where heating was insufficient to remove tissue, but where the temperature rise was sufficient to cause damage.

There is a great need for a laser system and method, which would be as cheap and safe as an infrared system, but would cut as cleanly and effectively as an ultraviolet system.

Currently, in the IR range, laser systems are used whose effect is based on water absorption, as water is the main component of soft tissue. Tissue is ablated, but the cuts are comparatively rough and exhibit thermal damage.

Tissue ablation was first shown to be potentially more effective (and cleaner) at 6.45 μm than at other infrared wavelengths by Edwards et al. (Edwards, G., Logan, R., Copeland, M., et al. (1994) “Tissue ablation by a free-electron laser tuned to the amide II band.” Nature, 371(6496), pp. 416-9.) using the Vanderbilt Free Electron Laser. They speculated that the infrared ablation efficiency is improved at this wavelength by vibrational interaction with the amide II protein band, from the second main component of soft tissue, collagen. This causes structural weakening of the tissue. However, as yet it is unclear whether the increased efficiency is due to this amide absorption alone or whether the pulse structure of a Free Electron Laser is a vital contributing factor. Recent studies (Uhlhorn, S. R., Mongin, D., Mackanos, M. A. & Jansen, E. D. (2001) in Tissue Interaction XII: Photochemical, Photothermal, and Photomechanical, Effects of IR wavelength on ablation mechanics: A study of acoustic signals. “Effects of IR wavelength on ablation mechanics: A study of acoustic signals” Ed. Jacques, S. L. Vol. 4257. Presented at Photonics West, SPIE, San Jose, Calif.) showed acoustic evidence to suggest that mechanical weakening forms a part of the ablation process at 6.45 μm but not at 2.94 μm. This was presented as being evidence that preferential absorption was involved. There is an order of magnitude difference in the absorption coefficient of soft tissue at 2.94 and 6.45 μm (Auerhammer, J. M., Walker, R., van_der_Meer, L. & Jean, B. (1999) “Dynamic Behaviour of photoablation products of corneal tissue in the mid-IR: a study with FELIX” Applied Physics B, 68, pp. 111-119.). A larger absorption coefficient causes a decreased extinction depth and consequently affects the ablative process. On the other hand, theoretical studies at the Lawrence Livermore National Laboratory have shown that there may be very large pressure waves generated by the micropulse train that could be responsible for the damage (Schwettman, H. & Crosson, E. R. (2000) in Proceedings of SPIE, Biomedical Applications of Free-Electron Lasers. “Mid-infrared ablation with single high-intensity picosecond pulses (oral presentation only)” Ed. Edwards, G. S. & Sutherland, J. C. Vol. 3925. Presented at Photonics West, SPIE, San Jose, Calif.; Uhlhorn, S. R., London, R. A., Makarewicz, A. J. & Jansen, E. D. (2000) in Proceedings of SPIE, Laser Tissue Interaction XI: Photochemical, Photothermal, Photomechanical. “Hydrodynamic modeling of tissue ablation with a free-electron laser” Ed. Jacques, S. L. Vol. 3914A. Presented at Photonics West, SPIE, San Jose, Calif.).

These theoretical studies may indicate that for efficient tissue ablation using infrared radiation the correct wavelength is not the only requirement and that also the specific pulse structure of laser may be essential. The difference in absorption may just amplify the effect of the pressure waves.

Tattoo Removal

The ablation of skin tissue and pigments has been investigated extensively over the past two decades leading to the wide acceptance of clinical procedures using the ns laser pulses to treat various pigmented lesions (X. H. Hu, W. A. Woodenb, M. J. Cariveau, Q. Fan, J. F. Bradfield, G. W. Kalmusd, S. J. Vole, Y. Sun, Tattoo Removal in Micropigs with Low-energy Pulses from a Q-switched Nd:YAG Laser at 1064 mL SPIE Proceedings, Vol. 4244 (2001)). A selective photothermolysis model (R. R. Anderson, J. A. Parrish, “Selective photothermolysis: precise microsurgery by selective absorption of pulsed radiation”, Science, 220, pp. 524-527 (1983) has been widely used to explain the experimental results obtained with the ns laser pulses using Q-switched lasers (R. E. Fitzpatrick, M. P. Goldman, J. Ruiz-Esparza, “Use of the alexandrite laser (755 nm, 100 ns) for tattoo pigment removal in an animal model”, J. Am. Acad. Dermatol., 28,745-750 (1993) T. J. Stafford, R Lizek, J. Boll, O. T. Tan, “Removal of colored tattoos with the Q-switched Alexandrite laser”, Plus. Reconstroc. Surg., 95, pp. 313-320 (1995)). Assuming a photothermal mechanism, the selective photothermolysis model has been successful in the elucidation of tissue ablation by long pulses of hundreds of microseconds in duration (R. G. Wheeland, “Clinical uses of lasers in dermatology”, Laser.s Surg. Med. 16, pp. 2-23 (1995)). However, a fundamental difference may exist between tissue ablation by long laser pulses and by ns pulses which exhibit strong electromagnetic fields because of the short duration. This strong field has not been widely appreciated in the clinical studies of skin tissue ablation. Considering a 10 ns pulse delivered with a laser fluence of 10 J/cm², the electric field strength by the laser pulse can rise up to 10⁷ V/m in the air. Such a strong electromagnetic field can cause ionization leading to breakdown in condensed materials including biological tissues (N. Bloembergen, “Laser-induced electric breakdown in solid”, IEEE. J. Quan. Electron., 10, pp. 375-386 (1974); D. Stern, R. W. Schoenlein, C. A. Puliafito, E. T. Dobi R. Biringruber, J. G. Fujimoto, “Corneal ablation by nanosecond, picosecond, and femtosecond lasers at 532 and 625 nm”, Arch. Ophthalrnol., 107,587-592 (1989)).

The ablation of pigmented lesions in the skin dermis by Q-switched lasers is accomplished with a large focal spot with a diameter of a few millimeters using the selective photothermolysis model. This, however, requires large pulse energies from a few hundred mJ to over 1 J from the Q-switched laser to operate above the ablation threshold, a costly requirement for incorporating diode-laser pumping into the Q-switched laser system. More importantly, the use of large pulse energy inevitably causes significant collateral tissue damage due to the mechanical and thermal actions by the pulse that often leads to excessive bleeding and scar formation. Therefore there is a great need to discover and develop new methods and devices to mitigate current existing problems with the ablation of the pigmented skin.

Drug Delivery with Nanoparticles and Light

A method of temperature-sensitive polymer/nanoshell composites for photothermally modulated drug delivery is disclosed by West et al. in the U.S. Pat. Nos. 6,428,811, 6,530,944. A thermally sensitive polymer-particle composite that absorbs electromagnetic radiation, and uses the absorbed energy to trigger the delivery of a chemical is described. Metal nanoshells are nanoparticulate materials that are suitable for use in the present composites and can be made according to a process that includes optically tuning or tailoring their maximum optical absorption to any desired wavelength primarily by altering the ratio of the core diameter to the shell thickness. Preferred nanoshells are selected that strongly absorb light in the near-infrared and thus produce heat. These nanoshells are combined with a temperature-sensitive material to provide an implantable or injectable material for modulated drug delivery via external exposure to near-IR light. The invention provides a means to improve the quality of life for persons requiring multiple injections of a drug, such as diabetes mellitus patients. However, this invention does not teach us how to use any size, shape and composite of nanoparticles for drug delivery without matching their absorption maxima with electromagnetic radiation wavelengths, and how to use surface plasmons generated in nanoparticles and nonlinear optical excitation for drug delivery. This invention also does not teach us how to use photothermally modulated drug delivery for the body surgery, tattoo removal and pigmentation removal, hair removal, and other biomedical applications.

Hair Removal.

Removal of unwanted hair is a common cosmetic concern. For hirsute women, treatment often requires drug therapy and various methods to physically remove the hair. Traditional methods of hair removal include shaving, waxing, tweezing, depilatory creams and electrolysis. Hair removal methods based on light technology, such as lasers and intense pulsed light systems, are alternative methods for longer-term hair removal. Intense pulsed light has been used in our clinic during the past years to treat light-to-dark skinned patients (F. Johnson and M. Dovale, Intense pulsed light treatment of hirsutism: case reports of skin phototypes V and VI J. Cutan Laser Therapy 1999; 1: 233-237). The use of lasers and intense pulsed light (IPL) sources for hair removal is based on the theory of selective photothermolysis (Anderson RR, Parrish JA. Selective photothermolysis: precise microsurgery selective absorption of pulsed radiation. Science 1983; 220: 524±7). A wavelength is chosen that will be maximally absorbed by a target chromophore to bring about the eventual destruction of the target structure with minimal damage to the surrounding tissue. With light based hair removal, the target chromophore is presumably the melanin produced by melanocytes in the hair matrix (Tse Y. Hair removal using a pulsed-intense light source. Dermatol Clin 1999; 17: 373±85). Melanin is also present in the epidermis, so that a wavelength must be chosen that will be maximally absorbed by the hair follicle. While melanin absorption is maximal at shorter wavelengths, longer wavelengths are necessary to penetrate hair residing deeper in the dermis. Additionally, the absorption coefficients of eumelanin (contained in brown and black hair) and phaeomelanin (contained in blonde and red hair) vary (i.e. at 694 nm, the absorbance of phaeomelanin is 30 times lower than that of eumelanin). Therefore, in order to optimize the results of hair removal for different body sites (where hair is located at various depths in the dermis) and different hair colors, both shorter and longer wavelengths (in the range of 600 to 1000 μm) may be necessary. An intense pulsed light source that generates 590±1200 nm non-coherent light pulses can be used with various cut-off filters to tailor treatment to the skin type and hair color of the patient. For treatment of dark-skinned individuals, higher cut-off filters can be used to omit light at lower wavelengths, where absorption of light in epidermal melanin is greatest. Longer pulse duration and longer wavelengths are available to target deeper structures, while protecting the epidermis. Additional protection of epidermal melanin is achieved by the use of multiple synchronized pulses separated by controlled delay times. Reports in the literature have demonstrated the safety of IPL hair removal treatment of various body sites for skin types I-V. Furthermore, large spot sizes allow many hair follicles to be targeted with each light pulse, thereby permitting quick treatment for large body areas. Presented here review of current methods and devices of hair removal indicates lack of enhancing hair removal techniques. Therefore, removing light hairs is much more difficult than removing dark hairs and this difficulty is usually compensated by more intense laser light that causing more damage to surrounding tissue.

Devices for Laser Surgery and Treatment.

The introduction of the laser into medicine has made many surgeries less invasive and more accurate and has even led to procedures that could not be performed before. Lasers require a delivery system to transport light to the surgical site. Currently, the most common delivery systems are based on optical fibers or waveguides. Fibers have even allowed lasers to reach areas previously unreachable with traditional surgery via the working channel of an endoscope or a catheter. But laser surgery poses a great many challenges like must be safe for both the surgeon and the patient, must withstand sterilization, bending, and the high laser power at specific wavelengths typically required for surgery. The major effort of current technology is to develop high power lasers and high photon throughput delivery systems operating at wavelengths close to the water absorption band of 2.9 μm and the tissue absorption bands in IR to precisely ablate tissue at the near cellular level with minimal thermal damage to adjacent healthy tissue.

These technological challenges would not be needed if new discoveries would allow for precise laser surgery at much lower laser power and at would not currently require specific wavelengths matching the water and the tissue absorption bands.

### SUMMARY OF THE INVENTION

Methods and devices for surface plasnion resonance-enhanced body surgery, tattoo and skin pigmentation removal, and body treatment are disclosed in the present invention. The methods of highly nonlinear interactions of surface plasmons of the embedded nanoparticles in the body with electromagnetic radiation and other forms of energy lead to the invention of enhanced and very confined body surgery, body treatment, drug delivery, tattoo and skin pigmentation removal, thrombosis, lithotripsy as described here. The enhancement may occur by few orders of magnitude and therefore most medical and cosmetic procedures can be performed at a much lower intensity of light and better-controlled surgery and treatment conditions. Another embodiment of the present invention is a method of making temporary, semi-permanent and permanent tattoos with a surface plasmon resonance technique.

The invention also describes endoscopes, catheters and other devices used for plasmon-enhanced body surgery and body treatment.

### FIGURES DESCRIPTION

FIG. 1. shows a metal nanoparticle coated with polymer, in which there are embedded drugs and biorecognitive sites.

FIG. 2. shows a complex of a metal nanoparticle and a tattoo ink molecule connected with chemical linker.

FIG. 3. shows several complexes between metal nanoparticles and tattoo ink molecules. Chemical linkers connect the nanoparticles with the ink molecules.

FIG. 4. shows a metal nanoparticles embedded to a skin tissue. The thermally/plasmons/ultrasound sensitive polymer has embedded tattoo ink molecules, which under surface plasmon resonance diffusing to the skin tissue.

FIG. 5. shows mixture of metal nanoparticles with tattoo ink molecules embedded to skin tissue.

FIG. 6. shows a catheter covered with a thin film of nanoparticles. The catheter is inserted to a targeted body. The nanoparticles and the targeted body are illuminated through the catheter and/or targeted body.

FIG. 7. shows embedded metal nanoparticles to a skin tissue with hairs. Nanoparticles there are close to follicles and under plasmon resonance absorption causing very efficient hair damage.

### DETAILED DESCRIPTION OF THE INVENTION

1. Abbreviations and Definitions

CW optical source—continuous waves source
SPR—surface plasmon resonance generated in a nanoparticle under illumination by electromagnetic radiation and other forms of energy
one-photon mode of excitation—process in which molecule is excited by a one photon absorption event
two-photon mode of excitation—process in which molecule is excited by simultaneous absorption of two photons
multi-photon mode of excitation—process in which molecule is excited by simultaneous absorption of three or more photons
step-wise mode of excitation—process in which molecule is excited by absorption of one photon and subsequently by absorption of second photon
up-conversion mode of excitation—process in which a molecule is excited by lower energy photon than energy of the lowest excited state of the molecule
nano island—a nanoparticle on a substrate without define shape
FRET—Fluorescence Resonance Energy Transfer

### 2. Exemplary Embodiments

Although the following detailed description contains many specifics for the purposes of illustration, anyone of ordinary skill in the art will appreciate that man y variations and alterations to the following details are within the scope of the invention. Accordingly, the following embodiments of the invention are set forth without any loss of generality to, and without imposing limitations upon, the claimed invention.

The present invention provides surface plasmon resonance (SPR) methods and devices for enhanced medical and cosmetic procedures. Nanoparticles embedded in a targeted body under irradiation of electromagnetic radiation, ultrasound, magnetic or other type of energy generate SPR enhanced interaction of embedded nanoparticles with the targeted body which can be applied to more effective tissue ablation, tattoo removal, skin pigmentation removal, photodynamic therapy, thrombosis, lithotripsy, cosmetic treatment, hair removal, wound healing, drug delivery.

The invention provides a novel methodology that overcomes limitations of conventional methods of using laser light and other forms of energy for animal or human body surgery and treatment.

The invention relates to the scientific reports of enhanced interaction between metal nanoparticles with molecules in the presence of surface plasmon resonance (M. Kerker, “Optics of colloid silver”, J. Colloid Interface Sci. 105, 298 (1985); Lakowicz et al, “Intrinsic fluorescence from DNA can be enhanced by metallic particles”, Biochem. Biophys. Res. Comm. 286, 875 (2001); Gryczynski et al., “Multiphoton excitation of fluorescence near metallic particles: enhanced and localized excitation”, J. Phys. Chem. B, 106, 2191 (2002)). In these reports, researchers used fluorophores (mostly organic laser dyes) to visualize or test the SPR enhanced interaction. Their studies show that the fluorescence intensity of the fluorophores can be enhanced by a factor as high as with one-photon mode excitation or ˜10⁸ with two-photon mode of excitation, and this enhancement occurs at distances up to 500 nm from metal nanoparticles (M. Moskovits: Rev. Mod. Phys. 57, 783 (1985); T. L. Haslett, L. Tay, M. Moskovits: J. Chem. Phys. 113, 1641 (2000), and references therein K. Kneipp, Y. Wang, H. Kneipp, L. T. Perelman, I. Itzkan, R. R. Dasari M. S. Feld: Phys. Rev. Lett. 78, 1667 (1997); Gryczynski et al., “Multiphoton excitation of fluorescence near metallic particles: enhanced and localized excitation”, J. Phys. Chem. B, 106, 2191 (2002)). The observed SPR enhanced interaction of fluorophores with metal nanoparticles was associated with intense photobleaching of fluorophores when fluorophores where at a distance 20 nm or less from metal nanoparticles (Ditlbacher H. et al., Appl. Phys. B 73, 373-377 (2001)).

This invention expands the above scientific findings to new methods and devices for a SPR enhanced interaction of embedded nanoparticles with biological substances and pigments in the body that are applied for enhanced medical and cosmetic procedures purposes. Biological substances considered in this invention are: a biomolecule, bacteria, living soft tissue, living hard tissue, abnormal tissue, abnormal cells, cells, virus, and other human body and animal body biological species. Pigments considered in this invention are all color tattoo inks and all natural body pigments. Embedded nanoparticles considered in this invention are: metal, metallic composite, metal oxide, metallic salt, electric conductor, electric superconductor, electric semiconductor, dielectric, quantum dot, metal-dielectric composite, metal-semiconductor composite, metal-semiconductor-dielectric composite.

In the presence of SPR, embedded nanoparticles interact with biological substances and pigments not only in direct contact with them, but also at nearby distances from nanoparticles, where exist very intense SPR electromagnetic fields (plasmons) (Ditlbacher H. et al., Appl. Phys. B 73, 373-377 (2001), raised temperature (Hirsch et al., PNAS, 100, 13549-13554 (2003), ultrasound (S. Coyle, et al., Phys. Rev. Let. 87(17), 176801, (2001)) and other type of generated energy. Under such enhanced interaction, biological substances and pigments can be destroyed. Such destruction of biomolecules and pigments can be used for more effective medical and cosmetic procedures and other applications, which will have positive impact on human health and economics.

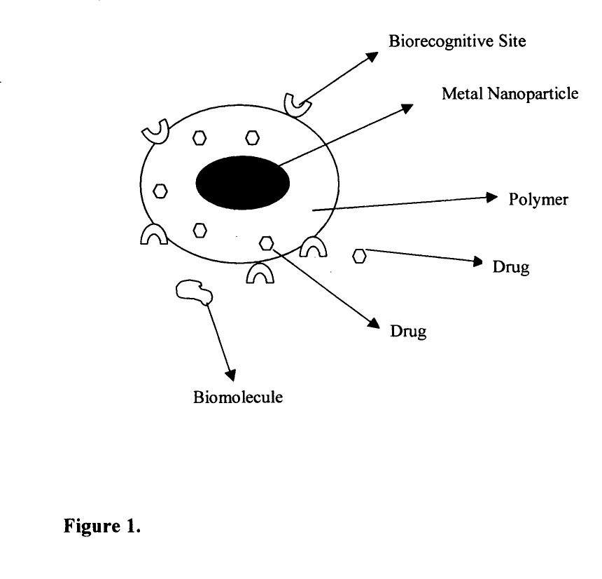

The proposed methods in the present invention take advantage of several positive properties of nanoparticles that can be used successfully in medical and cosmetic procedures proposed in the invention. 1) Metal nanoparticles display quadratic dependence of SPR generation on intensity of electromagnetic radiation and effect of nonlinear multiphoton excitation. These properties can be applied to three-dimensionally localized SPR enhanced medical and cosmetic procedures and other applications proposed in the invention. 2) Broadband structured absorption spectra of the nanoparticles from UVA to VIS/NIR are in the same spectral region as biological tissue components, which allow for much better SPR interaction with tissue and for more precise body ablation and body treatment, and faster healing process. 3) Nanoparticles are known as highly absorbed compounds and therefore they can be used to enhance ablation of soft tissue and hard tissue (e.g. tooth, bone). The ablation can be performed with one- and multiphoton excitations, where in the latter one, absorption bands of nanoparticles do not need to match with the wavelength of the laser. Hence, designing embedded nanoparticles sizes and shapes for different applications is not critical any more. One of ordinary skill in the art would appreciate that the scope of the present invention includes a method of SPR enhanced medical and cosmetic procedures at a specific location. The method is as follows. Nanoparticles coated with a biorecognitive polymer are retained in the specific location in the body and the generated surface plasmon resonance enhances the medical and cosmetic procedures at this body location. The biorecognitive polymer may have embedded a drug, which can additionally enhance the body treatment (FIG. 1). This method of localized body treatment can be applied to cancer treatment, cosmetic treatment, hair removal, drug delivery and wound healing.

Another embodiment of the present invention is the use of a nonlinear excitation SPR source irradiating embedded nanoparticles and the targeted body for medical and cosmetic procedures. The nonlinear source is generating SPR in embedded nanoparticles by nonlinear optical excitation processes like simultaneous absorption of two or three photons, step-wise excitation and/or up-conversion excitation. The targeted body has intrinsic chromophores which can be also nonlinearly excited at he same time as the SPR is generated. Therefore is expected enhanced interaction of surface plasmons of embedded nanoparticles with the targeted body chromophores that can lead to more chemical than mechanical body ablation and to more precise ablation. This nonlinearity of the SPR generation and chromophores excitation has big impact on other applications proposed in this invention, which are described in other embodiments. The use of linear one-photon excitation with nonlinear SPR generation in embedded nanoparticles is also a part of this invention. As was described earlier, in the SPR absorption process is not only generated heat, but also plasmons and ultrasound, which they very strongly interact with targeted body and significantly enhance body surgery and other applications proposed in the invention.

The invention uses electromagnetic radiation sources such as CW/pulsed and polarized/non-polarized light sources like lamps, LEDs, single and/or multiwavelength lasers for SPR enhanced body surgery and body treatment. SPR can also be generated by other techniques like sonic waves or electrical technologies, ultrasound, magnetic technologies and use for body surgery and other biomedical applications. Therefore these other techniques of generation SPR are considered as a part of the invention, particularly if these techniques are combined with optical techniques.

The embedded nanoparticles sizes can vary from subnanometers to micrometers. The sizes and shapes are designed for best SPR generation and interaction with biological substances. In the present invention the nanoparticles are embedded in the body or can be placed as a thin film on optical surfaces of devices, which are in direct contact with targeted body.

One of ordinary skill in the art would appreciate that the scope of the present invention includes a method of a very effective tattoo and skin pigmentation removal with nonlinear SPR and nonlinear optical illumination. As was published earlier by Patterson and Piston (George H. Patterson and David W. Piston, Photobleaching in Two-Photon Excitation Microscopy, Biophys J, April 2000, p. 2159-2162, Vol. 78, No. 4), the nonlinear optical excitation of two- or three-photon excitations cause substantial highly nonlinear photodecomposition of fluorescent dyes. This nonlinearity of the photodecomposition can be further increased by quadratic nonlinearity of SPR generation that leads to even more effective photodecomposition of fluorescent dyes or tattoo inks proposed in the present invention.

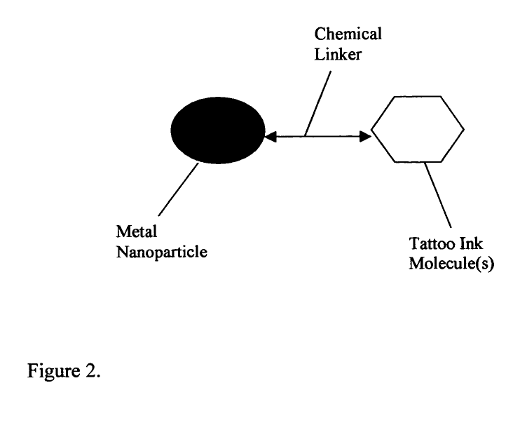

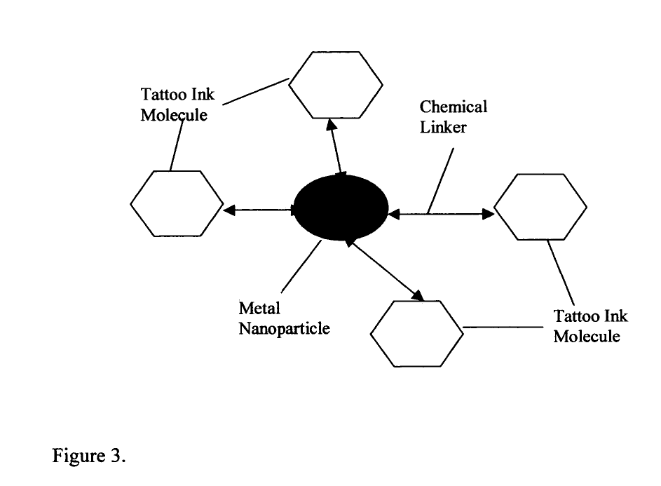

Another embodiment of this invention includes also a SPR method of using a complex of the metal nanoparticle and the tattoo ink molecule for making permanent, semi-permanent or erasable tattoos (FIG. 2, FIG. 3.). In the proposed method, easiness of removing tattoo ink from skin by the SPR method depends on a distance between the metal nanoparticle and tattoo ink molecule in this complex, e.g. at direct contact of metal nanoparticle with tattoo ink molecule, the tattoo can be much easier to remove from skin tissue than at the distance higher than 20 nm. The difficulties of removing tattoos increase with the longer distances between nanoparticles and ink molecules.

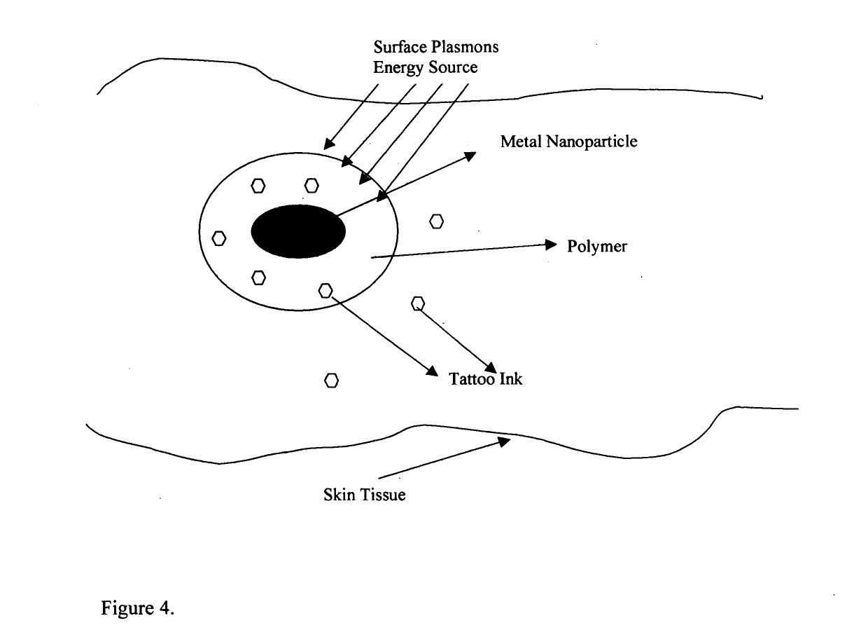

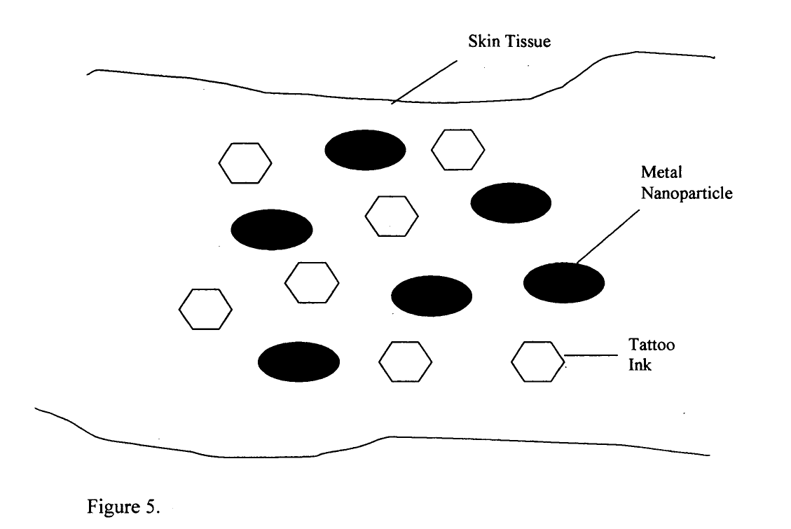

Another proposed method in this invention is to place tattoo ink into a polymer type material that is coating nanoparticles. In a process of removing a tattoo, light absorbed by metal nanoparticles will raise temperature of the polymer with embedded ink and ink will leak to the body and the body will digest it (FIG. 4). Any other energy source causing SPR generation and raising temperature of embedded nanoparticles and leaking tattoo inks from polymers to the body are considered as a part of the present invention. Mixtures of metal nanoparticles and tattoo ink molecules used for making tattoos are also a part of this invention (FIG. 5).

The present invention considers also the use of antibacterial properties of the metal nanoparticles during and after the plasmon enhanced medical and cosmetic procedures proposed in this invention. The metal nanoparticles could also be coated with biocide material which may additionally prevent the SPR enhanced surgery site from bacterial/microbial infections.

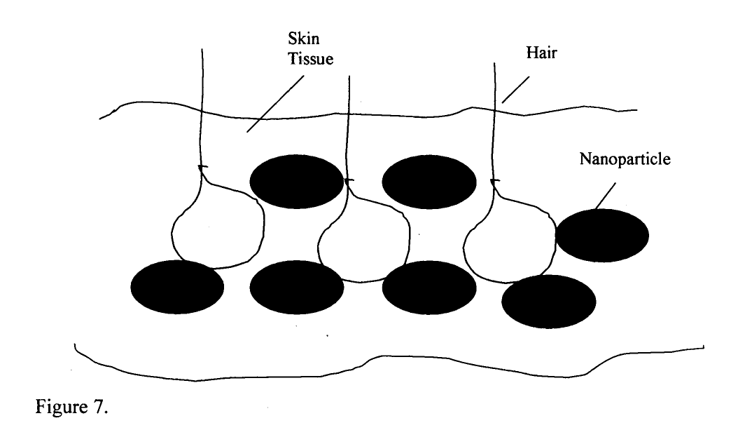

Another embodiment of this invention is a method of a SPR enhancing hair removal. The highly absorbed nanoparticles embedded to hair tissue, particularly hair follicles (FIG. 7), under illumination will generate plasmons, ultrasound and raise temperature, which very effectively will destroy hair follicles. The use of nonlinear SPR source of illumination will make this SPR enhancing hair removal method less invasive and further more matching wavelengths of laser light accordingly to hair color would be less critical. The nanoparticles coated with biorecognitive polymer for hair follicles are embedding in the targeted body closer to hair follicles that increases efficacy of the SPR enhancing hair removal.

Any suitable type of the device is included within the scope of the present invention, with all devices constructed from a surface plasmons generating source, directing/focusing optics to deliver light to a targeted body site and to monitor a feedback from the targeted body site, data acquisition and data analysis software, and electronics. The present invention includes the devices like a catheter, endoscope, fiber optics guide and light guide, lasers.

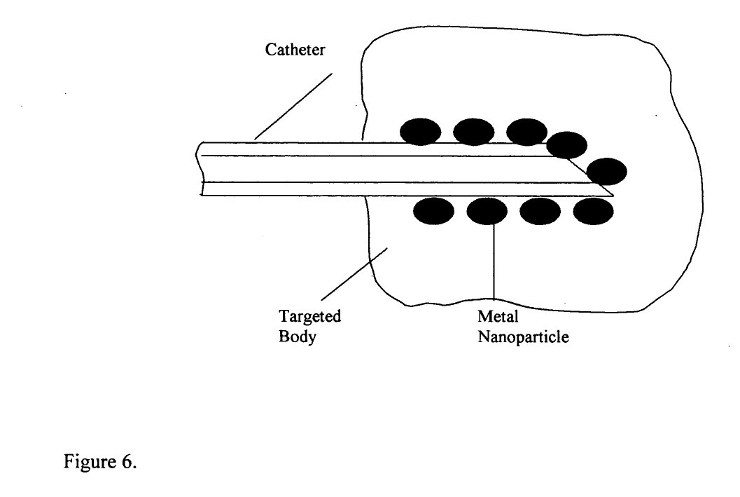

The devices optical parts, which are in direct contact with the targeted body, can be covered with a thin film of nanoparticles (FIG. 6) to SPR enhance medical and cosmetic procedures The placing nanoparticles on the device optical surface does not limit of using additional embedded nanoparticles in the targeted body to SPR enhance applications in the present invention.

1. A method and a device for a surface plasmon resonance enhanced body surgery, tattoo and skin pigmentation removal, hair removal, thrombosis, lithotripsy, drug delivery, photodynamic therapy and cosmetic body treatment comprising of:
a. a targeted body;
b. an embedded nanoparticle in said targeted body;
c. a nonlinear excitation surface plasmon resonance source exciting said embedded nanoparticle and irradiating said targeted body;
d. a surface plasmon resonance excited embedded nanoparticle causing biochemical changes in said targeted body;
e. a device is comprised of said nonlinear excitation surface plasmon resonance source, a delivery system of irradiation from nonlinear excitation surface plasmon resonance source to said targeted body and said embedded nanoparticles, a feedback system to monitor said targeted body, software and electronics.

2. A method and a device claimed in claim 1, wherein said targeted body is a human body, animal body.

3. A method and a device claimed in claim 1, wherein said targeted body is a tissue containing a tattoo ink, tissue containing pigmentation, abnormal tissue, soft tissue, hard tissue, cell, body fluid, non-cellular and non-tissue material.

4. A method and a device claimed in claim 1, wherein said embedded nanoparticle is a metal, metallic composite, metal oxide, metallic salt, electric conductor, electric superconductor, electric semiconductor, dielectric, quantum dot, metal and dielectric composite, metal and semiconductor composite, metal and semiconductor and dielectric composite.

5. A method of claim 4, wherein said embedded nanoparticle has optical absorption within the range of 200 nm to 10,000 nm.

6. A method of claim 4, wherein said embedded nanoparticle is an uncoated nanoparticle, coated nanoparticle.

7. A method of claim 6, wherein said coated nanoparticle has a coat made of a biorecognitive material, bioactive material, biological material, biocide material, dielectric material, chemorecognitive material, chemical active material, polymer, environmentally sensitive polymer, hydrogel, another layer of metal, another layer of semiconductor, another layer of dielectric, composition of metal and semiconductor and dielectric layers, polymer material containing a drug, polymer containing a tattoo ink, polymer containing a fluorescent marker, thermally sensitive material containing a drug, thermally sensitive material containing a tattoo ink, thermally sensitive material containing a fluorescent marker, thermally sensitive material containing a chemical substance.

8. A method and a device claimed in claim 1, wherein said embedded nanoparticle is a complex of said nanoparticle and a tattoo ink molecule bounded by a chemical linker of a length within a range of 0 nm and 10,000 nm.

9. A method and a device claimed in claim 1, wherein said embedded nanoparticle is a mixture of said nanoparticle and a tattoo ink molecule where a distance between them is within a range of 0 nm and 10,000 nm.

10. A method of claim 4, wherein said embedded nanoparticle size is within the range of 0.1 nm to 50,000 nm in at least one of the dimensions.

11. A method of claim 4, wherein said embedded nanoparticle is a thin film, colloid, fiber, nanoisland, nanowire, shell.

12. A method and a device claimed in claim 1, wherein said embedded nanoparticles are placed in said targeted body by an injection, ingestion, inhalation, adsorption, absorption, direct contact.

13. A method and a device claimed in claim 1, wherein said nonlinear excitation surface plasmon resonance source is a CW optical source, pulsed optical source.

14. A method of claim 13, wherein said optical source is selected from the group consisting of a laser, ion laser, semiconductor laser, Q-switched laser, free-running laser, fiber laser, light emitted diode, lamp, sun, fluorescence, electroluminescence.

15. A method of claim 14, wherein said optical source is a single wavelength polarized optical source at wavelength within the range of 200 nm to 10,000 nm, single wavelength unpolarized optical source at wavelength within the range of 200 nm to 10,000 nm.

16. A method of claim 14, wherein said optical source is a plurality wavelength polarized optical source at wavelengths within the range of 200 nm to 10,000 nm, plurality wavelength unpolarized optical source at wavelengths within the range of 200 nm to 10,000 nm.

17. A method of claim 14, wherein said pulsed optical source generates pulses at frequencies within the range of 1 Hz to 1 THz.

18. A method of claim 17, wherein said pulsed optical source generates an attosecond pulse, femtosecond pulse, nanosecond pulse, microsecond pulse, millisecond pulse.

19. A method and a device claimed in claim 1, wherein said nonlinear excitation surface plasmon resonance source is electromagnetic radiation, ultrasound, thermal energy, electrical energy, magnetic energy, electrostatic energy.

20. A method and a device claimed in claim 1, wherein said nonlinear excitation surface plasmon resonance source is irradiating said embedded nanoparticles and said targeted body with intensity within the range of 0.00005 mW/cm² to 1000 TW/cm².

21. A method and a device claimed in claim 1, wherein said nonlinear excitation surface plasmon resonance source generates surface plasmons in said embedded nanoparticles in a two-photon mode, multi-photon mode, step-wise mode, up-conversion mode.

22. A method and a device claimed in claim 1, wherein said nonlinear excitation surface plasmon resonance source generates surface plasmons in said embedded nanoparticles in a one-photon mode.

23. A method and a device claimed in claim 1, wherein said delivery system of irradiation from nonlinear excitation surface plasmon resonance source to said targeted body and said embedded nanoparticles is by a laser, fiber, waveguide, fiber and a contact tip, waveguide and a contact tip.

24. A method of claim 4 and claim 24, wherein said delivery system on a distal end directly contacting with said targeted body is covered with said thin film with nanoparticles.

25. A method and a device claimed in claim 1, wherein said feedback system is monitoring light, ultrasound, electric energy, magnetic energy and thermal energy from said embedded nanoparticles and said targeted body.

26. Methods of claim 25, wherein said light monitored from said targeted body is fluorescence, light scattering, fluorescence polarization, fluorescence spectrum, reflection, reflection spectrum, Raman spectrum, electroluminescence, bioluminescence, chemiluminescence.

27. A method and a device claimed in claim 1, wherein said device is a catheter, endoscope, laser surgery device.

28. A method and a device claimed in claim 1, wherein said embedded nanoparticles are used as an anti-bacterial agent in said targeted body.

29. A method and a device claimed in claim 1, wherein said surface plasmon resonance enhanced body surgery, tattoo and skin pigmentation removal, hair removal, thrombosis, lithotripsy, drug delivery, photodynamic therapy and cosmetic body treatment is a three-dimensionally localized.

32. Methods of claim 4, claim 7, claim 8 and claim 9 are used to make an erasable tattoo, semi-permanent tattoo, permanent tattoo.

33. Methods and a device claimed in claim 1 and claim 32 are used to remove an erasable tattoo, semi-permanent tattoo, permanent tattoo.
